(19) United States
(12) Patent Application Publication
(10) Pub. No.: US 2011/0142902 A1
(43) Pub. Date: Jun. 16, 2011

(54) Hypoxia Inducing Factor (HIF) Stabilising Glasses

(75) Inventors: Gavin Jell, Kent (GB); Robert Graham Hill, Maidenhead (GB); Molly Morag Stevens, London (GB); Maria Azevedo, London (GB)

(73) Assignee: IMPERIAL INNOVATIONS LIMITED, London (GB)

(21) Appl. No.: 12/994,463

(22) PCT Filed: May 27, 2009
(86) PCT No.: PCT/GB09/01323

§ 371 Date: Feb. 23, 2011

(30) Foreign Application Priority Data
May 27, 2008 (GB) 0809577.0
May 27, 2008 (GB) 0809578.8

(51) Int. Cl. A61F 2/00; A61K 9/14; A61K 33/42; A61K 8/46; A61Q 11/00; A61K 35/12; A61P 17/02; A61P 17/10; A61P 31/00; A61P 17/00; A61P 19/00

(52) U.S. Cl. 424/422; 424/423; 424/484; 424/604; 424/57; 424/93.7

(57) ABSTRACT

The present invention relates to a glass composition formulated to provide the controlled release of certain transition metal ions to regulate the cellular hypoxia pathway and the use of these hypoxia-pathway regulating glasses in medicine and in biomedical research, including in the repair, restoration or regeneration of diseased or damaged tissue.

The present invention relates to a glass composition formulated to provide the controlled release of certain transition metal ions to regulate the cellular hypoxia pathway and the use of these hypoxia-pathway regulating glasses in medicine and in biomedical research, including in the repair, restoration or regeneration of diseased or damaged tissue.

The oxygen pressure in tissues and organs of the body is important in determining growth, healing and cell behaviour (cell phenotype). Low oxygen pressure (hypoxia) in the body exists naturally in certain tissues (e.g. cartilage or bone marrow) and can be caused by a restriction of blood (and therefore oxygen supply) or by the increased metabolic demand of a developing tissue. Cellular responses to hypoxia through a hypoxia-sensing pathway are vital for natural healing following tissue damage and for new tissue formation. Hypoxia triggers a multifaceted adaptive cell-type specific response mediated by the heterodimeric transcription factor hypoxia-inducable factor 1 (HIF-1).

Under normoxia conditions the HIF-1 alpha subunit (HIF-1α) is degraded via ubiquitation and proteasomal digestion. In contrast, under hypoxic conditions HIF-1α degradation is inhibited, resulting in accumulation of the protein, dimerization with a β sub unit (HIF-1β), binding to hypoxia response elements (HREs) within target genes, and activation of transcription. This hypoxia pathway activates numerous genes and cellular mechanisms necessary for tissue repair and regeneration, including angiogenesis, lymphangiogenesis, cell differentiation, progenitor cell recruitment and enhancement of cytoprotective and bactericidal properties. These processes are crucial targets for a number of regenerative medicine strategies, including wound healing and tissue engineering and induction of the hypoxia pathway is an attractive route to promote these processes.

One of the tissue regeneration processes induced by the hypoxia pathway is angiogenesis (new blood vessel formation leading to the restoration of oxygen transport to ischemic tissues). Induction of angiogenesis is vital in certain tissue engineering strategies. Blood vessels supply the nutrients, oxygen, growth factors and cells vital for cell survival, extracellular matrix (ECM) remodelling and tissue regeneration. Long-term function, integration and survival of tissue engineered constructs is therefore dependent upon adequate vascularisation.

The growth of new tissue and tissue engineered constructs is not only dependent on new blood vessel growth but also on the right kind of blood vessels. A stable, mature fully differentiated vasculature needs to be developed, as opposed to “leaky” immature vessels, which can cause oedema and chronic inflammation. Administration of a single pro-angiogenic growth factor, e.g. vascular endothelial growth factor (VEGF), has been shown to promote the formation of leaky, immature vessels. There is therefore a need for an effective means of targeting the hypoxia response to cause the cellular production of a plethora of pro-angiogenic factors, leading to the formation of stable and mature blood vessels.

A further process induced by the hypoxia pathway is new lymphatic vessel growth (lymphangiogenesis). Impaired lymphatic function has been implicated in a number of pathological conditions including oedema and delayed wound healing. Hypoxia is known to induce the production of lymphatic growth factor VEGF-C and treatment with recombinant VEGF-C has been shown to accelerate wound healing, diabetic wound healing and reduce oedema. Regulation of the hypoxia pathway to promote lymphatic growth is therefore a desirable strategy for seeking to reduce oedema following reconstructive surgery.

A further effect of hypoxia is the enhancement of anti-microbial properties. Hypoxia gradients cause the recruitment of immune cells to ischemic tissues (e.g. wounds) and hypoxia increases the phagocytic activity of macrophages, thereby activating natural defence mechanisms and limiting potential infection. Fighting infection is vital for successful wound healing, including post-biomaterial implantation.

Furthermore, hypoxia influences the differentiation of a number of cell types including chondrocytes, neurons, adult stem cells, progenitor cells and embryonic stem cells. Thus, targeting of the hypoxia pathway can be used to direct the differentiation of certain cell lineages or maintain the phenotype of other cell types either in vitro or in vivo.

One therapeutic strategy to target the cellular response to hypoxia is recombinant and gene therapy technology. Whilst potentially effective, recombinant and gene therapy strategies typically involve challenging, lengthy and expensive isolation, manufacturing, purification and characterisation processes and the resulting therapeutic products generally have a relatively short shelf life.

An alternative strategy is to mimic hypoxia in tissues by the controlled release at precise concentrations of certain transition metal ions. These transition metal ions (including one or more of Co, Cu, Fe and Ni) are known to regulate the cellular hypoxia pathway (Maxwell, P. et al, Cancer Biol. Ther. 3(1):29-35 (2004)). Transition metal ions mimic hypoxia by preventing the destruction of the transcription factor HIF-1α in normoxic conditions and consequently causing the production of HRE related genes, including VEGF (U.S. Pat. No. 5,480,975).

The use of transition metal ions to regulate the cellular hypoxia pathway has huge potential in regenerative medicine as a way of stimulating tissue repair, creating tissue constructs and accelerating wound healing. There is therefore a need for an effective vehicle to allow controllable exposure of tissue to transition metal ions at a concentration and for a time sufficient to induce the hypoxia pathway.

Bioactive glasses are a group of silica or phosphate network based resorbable materials that have been shown to influence cellular behaviour and thereby tissue growth. The bioactivity of silicate glasses was first observed in soda-calcia-phospho-silica glasses in 1969. These glasses comprised SiO₂ (40-52%), CaO (10-50%), Na₂O (10-35%), P₂O₅ (2-8%), CaF₂ (0-25%) and B₂O₃ (0-10%). A particular example of a SiO₂—P₂O₅—CaO—Na₂O bioactive glass is manufactured as Bioglass®.

The bioactivity of bioactive glasses is the result of surface properties and the local release of dissolution ions in a physiological environment. The ionic products of bioactive glass (e.g. Bioglass®) dissolution have been shown to regulate the expression of genes important for both hard and soft tissue formation.

Many bioactive silica glasses are based on a formula called ‘45S5 Bioglass®’, signifying 45 wt % silicon dioxide (SiO₂), and a 5:1 molar ratio of calcium (Ca) to phosphorus (P). However, variation in the ratio of these components, and inclusion of other components such as boron oxide (B₂O₃), magnesium oxide (MgO), potassium oxide (K₂O), strontium oxide (SrO) and calcium fluoride (CaF₂), has allowed modification of the properties of the bioactive glass, including the rate of dissolution and apatite formation.

The commercially available 45S5 Bioglass® composition, comprising SiO₂, Na₂O, CaO and P₂O₅ has been shown in vitro to have an effect in inducing VEGF upregulation (WO2004/071542). However, the observed increase in VEGF production is marginal, the glasses do not regulate the hypoxia pathway and do not have a composition modified in order to induce VEGF production and/or generate an angiogenic response.

It has now been determined that by manipulation of the chemical composition, concentration, pore size and manufacturing method or biologically compatible and/or bioactive glasses, a glass can be produced which provides controlled release of hypoxia stimulating ions (Cu, Ni, Fe, and/or Co) at physiologically active levels and that this glass can be used to beneficially mimic hypoxia, stabilize the transcription of HIF-1α and induce the hypoxia response (e.g. hypoxia gene expression) in normoxia conditions. A glass which targets the hypoxia response by the controlled release of hypoxia mimicking ions will advantageously stimulate not only VEGF expression but also a host of other factors important in angiogenesis and other responses induced by the hypoxia pathway and important in tissue regeneration. In addition to regulating the hypoxia pathway the glass chemical composition can be optimised to activate other important biological processes important for tissue regeneration and implant integration (e.g. apatite formation in bone tissue regeneration)

Therefore, in a first aspect the present invention provides a glass formed from:

30-62% SiO₂ (for example 45-62%, 47-53% or 47-50% SiO₂);

### 0.5-15% P₂O₅;

a combined content of CaO and SrO of 12-45%;
a combined content of Na₂O and K₂O of 6-30%; and
0.1-10% of a source of hypoxia mimicking ions,
wherein the hypoxia mimicking ions are selected from one or more of Co, Cu, Mn, Ni and Fe ions.

In certain embodiments, the SiO₂ content is 45-62%, preferably 47-53%, more preferably 47-50% and/or the P₂O₅ content is 0.5-1.5% and/or the combined content of Na₂O and K₂O of 6-28%.

In certain embodiments a glass of the invention additionally comprises one or more of a source of Mg (eg MgO) at 0-12%, a source of Zn (eg ZnO) at 0-10%, a source of Boron (eg B₂O₃) at 0-15% and a source of fluorine (eg CaF₂) at 0-10%.

The glass preferably comprises an amorphous glass network and the hypoxia mimicking ions are integrated into the amorphous glass network. Preferably, crystalline structure is absent.

The composition of the glass is essential in order for hypoxia mimicking ions to be incorporated into the amorphous network of the glass and for the glass, in use in a physiological environment, to provide controlled release of the ions. In combination with controlled delivery of hypoxia ions, the chemical composition of a glass influences the physical and chemical properties of the glass. The chemical composition of a glass of the invention can therefore be modified for the desired application and tissue type. Preferred compositions are detailed below.

The percentage contents of the glass composition as referred to throughout are molar percentages. Metal oxides used in formation of the glass composition, for example CaO, provide a source of the respective metal ions. Where a glass is recited as being formed from or comprising a certain percentage of an oxide, during formation of the glass, the oxide itself may be provided or alternatively a compound that decomposes to form the oxide may be provided. Accordingly, the source of hypoxia mimicking ions may be CoO, CuO, MnO, NiO, an iron oxide or a mixture thereof.

Advantageously, the composition of the glass is suitable to provide in vivo release of the hypoxia mimicking ions at a level suitable to induce the hypoxia pathway. In a preferred embodiment, the hypoxia stimulating ions are Co or Cu. More preferably, the hypoxia stimulating ions are Co ions.

In a preferred embodiment, the source of hypoxia stimulating ions is present at 0.2-10 mol %, preferably 0.5-5 mol %, more preferably 0.5 to 4 mol %, even more preferably 1-3 mol % or 2-4 mol %.

In highly disrupted glass networks (such as bioactive glasses) transition metal ions are known for use as nucleation agents and cause crystallisation. This results in an inherent loss of homogeneity causing lack of predictability. Advantageously, the compositions of this invention are tailored to avoid this effect and instead provide for controlled release of transition metal ions.

Advantageously, a glass of the present invention provides controlled release of hypoxia stimulating transition metal ions. Controllable hypoxia release rates are of critical importance for regulating the hypoxia pathway. Whilst many transition metals are important dietary requirements, toxicity may occur in local tissue or systemically at high concentration and/or long-term exposure to transition metal ions. For example, whilst cobalt is a vital component of vitamin B12, high cobalt levels have been associated with cell death in vitro and in vivo. Persistent long-term local tissue exposure to hypoxia pathway mimetics may also cause chronic inflammation and associated pathologies.

The local physiological concentration of hypoxia mimicking ions is dependant upon glass composition, transition metal type, application and target tissue. The composition of the glass is vital for controlled release. Only with specific compositional ranges can controlled release at physiological active and not pathological ranges occur. For example, a highly cross linked glass would prevent hypoxia ion release, whilst a highly disrupted glass would release too much hypoxia ion too quickly. In a preferred embodiment, a glass of the present invention is formulated to provide a local concentration of transition metal ions of between 0.1 μM-500 μM and for a period of 0-31 days.

In some embodiments, the glass is formed from: 47-50% SiO₂; 0.5-15% P₂O₅ (preferably 0.5-1.5%); 0-2% B₂O₃; a combined molar percentage of CaO and SrO of 18-25%; a combined molar percentage of Na₂O and K₂O of 24-27%; 0-2% ZnO; 0-2% MgO and 0.1-10% (preferably 0.5-10%, more preferably 0.5-5%) hypoxia stimulating ions. A glass of this composition may be for use in bone/hard-tissue applications, such as a bone-regeneration material.

In other embodiments, the glass is formed from: 49-50% SiO₂; 0.5-15% P₂O₅ (preferably 0.5-1.5%); a combined molar percentage of CaO and SrO of 16-18%; a combined molar percentage of Na₂O and K₂O of 18-20%; 1-10% a source of fluorine (eg. CaF₂); 1-3% ZnO; 1-3% MgO and 0.1-5% (preferably 1-3%) hypoxia stimulating ions. A glass of this composition may be for use in periodontal applications.

In yet other embodiments, the glass comprises 46 to 50% SiO₂, 0.5-15% P₂O₅ (preferably 0.5-1.5%), 0 to 2% B₂O₃, 8 to 40% CaO (preferably 8-27%), 0 to 15% SrO, 5 to 7% Na₂O, 4 to 7% K₂O, 0-4% ZnO (preferably 2-4%), 0-4% MgO (preferably 2-4%), 0 to 9% CaF₂ and up to 5% of a source of hypoxia mimicking ions. Preferably, the glass comprises 47-50% SiO₂, 0.5-1.5% P₂O₅ (preferably approximately 1%), 8-27% CaO, 3-15% SrO, approximately 3% ZnO, approximately 3% MgO, and approximately 2% CoO, CuO or NiO. The combined molar percentage of ZnO, MgO, CoO, SrO and P₂O₅ within the glass may be 1-12%. The glass may be formed from: 46-50% SiO₂ (preferably 47-50%); 0.5-15% P₂O₅ (preferably 0.5-1.5%); 0-2% B₂O₃; a combined molar percentage of CaO and SrO of 20-29%; a combined molar percentage of Na₂O and K₂O of 12-14%; 2-4% ZnO; 2-4% MgO; 0-9% CaF₂ and 0.1-5% (preferably 1-3%) hypoxia stimulating ions. In ceratin embodiments, the glasses may comprise 46 to 50% SiO₂, 0.5-1.5% P₂O₅, a total molar percentage of CaO, ZnO, MgO and SrO of 35-40%, 5-7% Na₂O and 5 to 7% K₂O. The glass compositions described above are preferably glasses for use in forming a porous sintered scaffold for bone. In a preferred embodiment, the glass is thus provided as a porous sintered scaffold comprising ions that mimic hypoxia. Such scaffolds are useful for bone regeneration and repair.

In yet another preferred embodiment, the glass is formed from: 48-50% SiO₂; 0.5-15% P₂O₅ (preferably 0.5-1.5%); a combined molar percentage of CaO and SrO of 30-41%; a combined molar percentage of Na₂O and K₂O of 6-8% and 0.1-10% (preferably 1-3%) hypoxia stimulating ions. A glass of this composition may be for use as a filler for composites. The glass may additionally comprise 4-6% ZnO and 4-6% MgO and where these components are present the glass may be particularly suitable for use as a filler for non-bone composites.

In yet other embodiments, the glass is formed from: 47-62% SiO₂; 0.5-15% P₂O₅ (preferably 0.5-1.5%); 0-2% B₂O₃; a combined molar percentage of CaO and SrO of 12-17%; a combined molar percentage of Na₂O and K₂O of 12-27%; 0-8% ZnO (preferably 0-6%, more preferably 3-6%); 0-8% MgO (preferably 0-3%) and 0.1-10% (preferably 0.5-5%) hypoxia stimulating ions. A glass of this composition may be for use in soft tissue applications.

For soft tissue applications, the glass is biologically compatible, but preferably not bioactive. One way of avoiding bioactivity is to increase the SiO₂ content, for example a content up to 62%, such as 55-62%. Another way of avoiding apatite formation is to increase MgO or ZnO concentration. Thus, in ceratin embodiments, the glass comprises 4-8% ZnO or MgO.

In yet other embodiments, the glass may be formed from: 47-62% SiO₂ (preferably 47-50%) 0.5-15% P₂O₅ (preferably 0.5-1.5%); 0-2% B₂O₃; a combined molar percentage of CaO and SrO of 18-21%; a combined molar percentage of Na₂O and K₂O of 24-27%; 0-6% ZnO (preferably 3-6%) and 0.1-5% (preferably 2-5%) hypoxia stimulating ions. A glass of this composition may be for use in a shampoo to treat alopecia. The glass is preferably a bioactive glass.

In yet other embodiments, the glass may be formed from: 30-50% SiO₂, 0.5-15% P₂O₅ (preferably 0.5-11%); a combined molar percentage of CaO and SrO of 22-28% (this may be solely CaO, solely SrO or a combination thereof); 22-28% Na₂O and 0.1-5% (preferably 1-3%) hypoxia stimulating ions. Glasses of this composition are pH stable and thus particularly useful in applications where minimising pH change can important, for example in cell culture systems or shampoo or for topical administration. When administered topically, such glasses may act via the HIF-1 pathway to provide enhanced wound repair (dermal-subdermal) or, in the case of use in shampoos, to combat alopecia by increasing follicle VEGF expression.

Stimulation of the hypoxia pathway provides a number of advantages over alternative strategies commonly used in the art to stimulate angiogenesis, i.e. the recombinant release of angiogenic factors. A glass according to the present invention has advantages over the use of materials, gels and scaffolds which incorporate recombinant proteins or gene transfer technologies to stimulate angiogenesis both in terms of efficacy and economics.

In contrast to recombinant proteins or gene transfer, a glass of the present invention requires a relatively “low technology” manufacturing processes, requires inexpensive raw materials and has an extremely long shelf life. Furthermore, glasses do not have the safety concerns associated with the use of vectors for gene transfer.

In a preferred embodiment, the glass, when in contact with living tissue (or cells/organoids in vitro), elicits a favourable biological response. A favourable biological response, namely induction of the hypoxia pathway, is caused by the release of hypoxia pathway regulating ions.

The glasses of the invention are biologically compatible. In certain preferred embodiments, the glass is a bioactive glass. In alternative preferred embodiments, the glass compositions are not bioactive (but still biocompatible). It is undesirable to stimulate mineralisation in soft tissues and this is a major drawback of existing compositions for soft tissue applications.

In silica based glasses, SiO₂ forms the amorphous network of the bioactive glass, and the molar percentage of SiO₂ in the glass affects its Network Connectivity (NC). NC is the average number of bridging bonds per network forming element in the glass structure. NC determines glass properties such as viscosity, crystallisation rate and degradability. At a NC of 2.0 it is thought that linear silicate chains exist of infinite molar mass. As NC falls below 2.0, there is a rapid decrease in molar mass and the length of the silicate chains. At an NC above 2.0, the glass becomes a three dimensional network. For the glass to be degradable and able to form apatite, NC must be below 2.6, or more preferably below 2.4. For applications in bone where apatite formation is important the bioactive glass therefore has a network connectivity of 2.6 or less, preferably 2.4 or less. Increasing SiO₂ content to raise network connenectivity to 2.6 or more can reduce or remove apatite forming ability.

Network connectivity is calculated for this invention according to the method set out in Hill R. J. Mat. Sci. Letts. 1996 Jul. 1; 15(13):1122-5, but with the assumption that the phosphorus is considered to exist as a separate orthophosphate phase and is not as part of the glass network.

In a preferred embodiment, the glass of the invention is resorbable under physiological conditions. Accordingly, the invention encompasses resorbable glasses. One aspect of this invention details hypoxia mimicking dissolvable glasses which cause a protective effect on certain cell types which will be important in sustaining the viability of tissue engineered constructs both during transit, during surgery and post-implantation.

In a preferred embodiment, the glass composition comprises a source of one or more of Li, Mg, Zn, B or F.

Glasses of the invention may comprise one or more components selected from a source of calcium, phosphate, magnesium, strontium, zinc, boron, fluorine or an alkali metal such as sodium or potassium. Preferably these components are provided as compounds including, but not limited to, Na₂O, Na₂CO₃, NaNO₃, Na₂SO₄, sodium silicates, K₂O, K₂CO₃, KNO₃, K₂SO₄, potassium silicates, CaO, CaCO₃, Ca(NO₃)₂, CaSO₄, calcium silicates, MgO, MgCO₃, Mg(NO₃)₂, MgSO₄, magnesium silicates, ZnO, ZnCO₃, Zn(NO₃)₂, ZnSO₄, and zinc silicates, CoO, CO₂O₃, CoCO₃, Co(NO₃)₂, CoCl₂, CoF₂, CoSO₄, cobalt silicates, NiO, Ni₂O₃, NiCO₃, Ni(NO₃)₂, NiSO₄, NiCl₂, NiF₂, nickel silicates, CuO, CuCO₃, Cu(NO₃)₂, CuCl₂, CuF₂, CuSO₄, copper silicates and any such compounds that decompose to form an oxide.

Where glasses of the invention are referred to above as being formed from or comprising certain components, it will be appreciated that the glass is formed from these components, but that additional components may also be present within the glass network. However, the invention does therefore also encompass glasses having the glass compositions as described herein, where no additional components are present within the glass network i.e. glasses “consisting essentially of” the described components. For example, glasses of the invention may be aluminium-free. The glasses may also be free of elements such as silver and the like.

It will be appreciated that the exact molar percentage of the components of the glass affects the physical and biological properties of the glass. Different uses of the glass require different properties, and hence the properties of the glass may be tailored to a particular intended use by adjusting the molar percentage of each component. For example, the chemical composition of glasses can be tailored for specific applications, for example reducing or removing apatite forming ability for non-bone applications by increasing Si, Zn and/or Mg concentration. Furthermore, whilst the hypoxia response is ubiquitous to all cells, the type of response is cell specific, allowing creation of a glass tailored to the structural and mechanical properties of a target tissue.

In a preferred embodiment, the glass comprises a source of calcium. For the purposes of this invention, a source of calcium includes calcium oxide or any compound that decomposes to form calcium oxide. The presence of a source of calcium in the glass leads to release of Ca²⁺ ions from the surface of the glass, which aids and increases the rate of formation of a calcium phosphate-rich layer on the surface of the glass. The formation of this layer is an important step in the generation of bone tissue and a bioactive glass comprising calcium is therefore particularly suitable for use in promoting bone tissue repair and regeneration. It should be appreciated that the calcium phosphate-rich layer can form without the provision of calcium ions by the bioactive glass, as body fluid itself contains calcium ions. Thus, for the purposes of this invention, bioactive glasses containing no calcium can be used. Preferably, the molar percentage of the source of Ca (eg CaO) is 0% to 45%, more preferably 10% to 40%.

The glasses of the present invention comprise P₂O₅. Whilst hydroxycarbonated apatite can form without the provision of phosphate ions by the bioactive glass, as body fluid itself contains phosphate ions, the provision of phosphate ions by the bioactive glass increases the rate of formation of hydroxycarbonated apatite. In addition, the provision of P₂O₅ has a beneficial effect on the viscosity-temperature dependence of the glass, increasing the working temperature range which is advantageous for the manufacture and formation of the glass.

The glass of the present invention preferably comprises a source of magnesium including but not limited to MgO, MgCO₃, Mg(NO₃)₂, MgSO₄, magnesium silicates and any such compounds that decompose to form magnesium oxide. Magnesium ions decrease the size of the hydroxycarbonated apatite crystals formed and decrease the thermal expansion coefficient. Reduced apatite crystal size thereby reduces the formation of brittle bone. Preferably, the molar percentage of MgO is 0% to 12%, more preferably 0% to 10%. A portion or all of the magnesium can be provided as magnesium oxide.

The inclusion of zinc ions (molar percentage of ZnO is 0% to 5%) also decreases the size of the hydroxycarbonated apatite crystals formed and decreases the thermal expansion coefficient. Decreasing the thermal expansion coefficient is advantageous when the glass is intended for use as a coating. The glass can be introduced into a bone fracture or a damaged region of bone. Accordingly, glasses of the present invention may be formed from a source of zinc, included but not limited to ZnO, ZnCO₃, Zn(NO₃)₂, ZnSO₄, and zinc silicates and any such compounds that decompose to form zinc oxide. Preferably, the molar percentage of the source of zinc (determined as ZnO) is 0-10%. A glass comprising a source of zinc is particularly useful for promoting soft tissue repair and regeneration, in applications such as wound healing, directing stem cell differentiation and cartilage tissue repair. The incorporation of zinc into the glass of the present invention promotes wound healing and aids the regeneration of diseased or damaged tissue. Without being bound by theory, research suggests that, within certain compositions, the inclusion of a source of zinc ions (ZnO) above 4%-5% molar percent inhibits apatite formation, which is preferable for non-bone applications. Research suggests that as ZnO content is increased above a molar percentage of ZnO of 4%-5%, its role with the glass switches from that of a network modifier to an intermediate oxide thus creating a more stable glass structure and preventing the ion exchange necessary for apatite formation. Therefore, in a preferred embodiment the invention provides a glass comprising a source of zinc ions at a molar percentage of above 5%. This glass is of particular use for soft tissue applications.

Similarly, high MgO content can be employed to knock out bioactivity and provide a glass suitable for soft tissue applications. For example CaO and SrO can be replaced by MgO and/or ZnO to knock out bioactivity. Zn²⁺ and Mg²⁺ act by increasing the NC of the glass and reducing glass degradation/dissolution. Zn²⁺ and Mg²⁺ may also block planes in the apatite crystal lattice and inhibit crystal growth of the apatite.

In another preferred embodiment, the glass of the present invention comprises a source of boron, preferably as B₂O₃. As with P₂O₅, B₂O₃ is believed to have a beneficial effect on the viscosity-temperature dependence of the glass, increasing the working temperature range which is advantageous for the manufacture and formation of the glass. B₂O₃ is also believed to increase the size of the processing window between the glass transition temperature of the bioactive glass and the onset temperature for crystallisation, allowing the sintering of glass powders without crystallisation. This is advantageous as the formation of crystals in the bioactive glass generally decreases its bioactivity. Preferably, the molar percentage of B₂O₃ is 0% to 15%. More preferably, the molar percentage of B₂O₃ is 0% to 12%.

The glass of the present invention preferably comprises a source of fluorine, preferably, in the form of one or more of CaF₂, SrF₂, MgF₂, NaF or KF. Fluoride stimulates osteoblasts, and increases the rate of hydroxycarbonated apatite deposition. Fluoride and strontium function synergistically in this regard. Fluoride also promotes the formation of more mixed-type apatite structures with a greater similarity to natural biological forms by substituting readily for hydroxyl ions in the apatite lattice. The mixed apatite is more thermodynamically stable and therefore less soluble and less resorbable. Fluoride can also be used to decrease the melting temperature of the bioactive glass. Preferably, the fluorine is provided in a molar percentage of 0% to 50%, more preferably 0% to 25%. Preferably, the source of fluorine (preferably CaF₂) is provided in a molar percentage of 0% to 10%, or 1% to 7%. Preferably at least 1% is present.

In some preferred embodiments of the glasses described herein, the combined molar percentage of SiO₂, P₂O₅ and B₂O₅ does not exceed 80%.

Furthermore, in the combined molar percentage of SrO, CaO, MgO, Na₂O and K₂O in glasses of the invention may be 40-60%.

Depending upon its intended use, the glass may be provided in particulate form, as 3-D structure or as a solid such as a disk or monolith. In particular, the glass can be provided in any required shape or form, for example as a pellet, sheet, disk, foam, etc. In particulate form, the preferred particle size depends upon the application of the bioactive glass in question, however preferred ranges of particle sizes are less than 1200 microns, preferably between 1 and 1000 microns, more preferably 50 to 800 microns, more preferably 100 to 700 microns. The range of particle size required depends upon the application and the bioactivity of the glass. For example, fillers for composites or for sintered glasses would be provided with a particle size of 45 microns or less. In particulate form, such as a powder, the glass may be included in a cement, a paste or a composite. The glass may be included (for example as a filler) in substances including but not limited to acrylic, bisphenol A diglycidylether methacrylate (Bis GMA) and polyactide. The glass powder may be sintered to create coatings or to form a porous solid for use as a scaffold. In addition, the glass may be incorporated into a degradable polymer scaffold. The glass may be in the form of granules.

The glass of the invention may also be used to form porous hypoxia pathway regulating scaffolds using a gel cast foaming method. This gel cast method enables the manufacture of hypoxia stimulating biocompatible porous scaffolds. This scaffold has unique properties whereby it can act as template for bone growth in three dimensions, has the appropriate mechanical properties for bone regeneration in load bearing sites, is degradable at a controlled rate, contains a source of calcium ions to provide bioactivity, can stimulate blood vessel growth, can stimulate bone growth and is capable of commercial production and sterilisation for clinical use. The gel cast foaming technique involves the foaming a glass particulate slurry with a surfactant and in situ polymerisation of gelling agents. The gelled foam can then be poured into a mould immediately prior to gelation and heat treated to remove the polymer and sinter the glass particles. Accordingly, a glass of the invention may be provided as a porous sintered scaffold comprising ions that mimic hypoxia and the invention therefore encompasses a porous sintered scaffold comprising a glass of the first aspect of the invention. In a preferred embodiment, the invention provides a porous bioactive scaffold containing concentration gradients of hypoxia mimicking ions. Such a scaffold can be formed by direct laser sintering and multi-layer sintering of melt-derived porous scaffolds. These ionic release gradients will mimic the natural in vivo hypoxia gradients and thereby cell signalling concentration gradients for cell migration and recruitment.

The glass is preferably provided as a melt-derived glass. The melt-derived glass can further be sintered using known technology. The melt-derived glass is preferably prepared by mixing and blending grains of the appropriate carbonates or oxides, melting and homogenising the mixture at temperatures of approximately 1250° C. to 1500° C. Homogenisation is preferably performed by oxygen bubbling. The mixture is then cooled, preferably by casting of the molten mixture into a suitable liquid such as deionised water, to produce a glass frit.

The glass chemical composition and form will depend upon the application. The hypoxia pathway regulating bioactive glasses can be used in particulate form, as a monoliths, 3D porous scaffolds, fibres and/or coatings mentioned forms or hypoxia mimicking glasses incorporated into or onto implanted materials, tissue regeneration constructs and wound healing devices, such as tissue engineering scaffolds, sutures, prosthetic implants, polymer matrixes, fibrin gels, hydrogels, plasters, wound dressings, creams, shampoo and the like. The hypoxia stimulating glass compositions can also be used in devices used for in vitro and ex vitro cell culture. Furthermore, the glasses could be used elicit certain cell responses in vitro prior to therapeutic use of cells in vivo.

A glass of the present invention can be incorporated into another material, for example a hydrogel, a gel, a cream, a scaffold and/or a polymer composite. The incorporation of a glass into another material makes it possible to take advantage of the glass' ion release properties for a number of regenerative medicine applications.

Hydrogels obtained by cross-linking of water soluble polymers, e.g., cross-linked polyacrylamide gels or polysaccharide gels are particularly preferred. Polysaccharide hydrogels that are preferred include alginate, carrageenan, agar, and agarose; other polysaccharides, e.g., curdlan, pullulan, gellan and the like are also useful as the hydrogel-forming component.

In a further preferred embodiment, the glass is provided as a composition for topical application, for example, to treat a wound or burn, for use in skin grafting, in which the composition is applied to a graft site prior to application of the donor tissue, or applied to the donor tissue itself, or for use in surgery, applied to a surgical site to minimise post-surgical oedma and infection at the surgical site whilst promoting wound healing.

The composition of the present invention may comprise glass in the form of glass particles. The glass particles may be provided alone, or in combination with additional materials, including but not limited to antibiotics such as erythromycin and tetracycline, antivirals such as acyclovir and gancyclovir, healing promotion agents, anti-inflammatory agents such as corticosteroids and hydrocortisone, immunosupressants, growth factors such as basic fibroblast growth factor, anti-metabolites, cell adhesion molecules, bone morphogenic proteins, vascularising agents, anti-coagulants and topical anaesthetics such as benzocaine and lidocaine.

In a second aspect, the present invention provides a glass as described above for use in medicine, preferably for use in the prevention and/or treatment of damage to a tissue. For the purposes of this invention, the tissue can be bone tissue, skin, cartilage, soft tissues including connective tissues and dental tissues including calcified dental tissues such as enamel and dentin. The tissues can be animal tissues, more preferably mammalian or human tissues.

In a preferred embodiment, the glass is provided for use in inducing angiogenesis or lymphangiogenesis, promoting anti-microbial activity or sustaining cell viability.

Throughout this text, the prevention and/or treatment means any effect which mitigates any damage or any medical disorder, to any extent, and includes prevention and treatment of damage itself as well as the control of damage. The term “treatment” means any amelioration of disorder, disease, syndrome, condition, pain or a combination of one or more thereof. The term “control” means to prevent the condition from deteriorating or getting worse for example by halting the progress of the disease without necessary ameliorating the condition. The term “prevention” means causing the condition not to occur, or delaying the onset of a condition, or reducing the severity of the onset of the condition.

In particular, the terms prevention and/or treatment include the repair and/or reconstruction of tissue. For the purposes of this invention, the term “repair” means the restoration of the tissue to a condition of working order for example by the in vivo stimulation of biological processes. The term “reconstruction” means the rebuilding of the tissue and includes the temporary or permanent incorporation into the tissue of an external component such as a scaffold, model etc.

For the purposes of this invention the damage can be mechanical damage, can be caused by an external agent or can be a result of an internal biological process. Examples of mechanical damage include damage caused by trauma, surgery, age related wear, etc. Examples of damage caused by an external agent include damage caused by a medicament, a toxin, or a treatment regime (such as chemotherapy or radiotherapy), for example dialysis-related amyloidosis, damage caused by diseases such as a bacterial, viral or fungal infection, such as osteomyelitis, a genetic condition such as osteogenesis imperfecta and hypophosphatasia, inadequate nutrition, age-related disorders, a degenerative disorder or condition such as osteoporosis and bone cancers including osteosarcoma and Ewing's sarcoma. Examples of damage caused as a result of an internal biological process include an autoimmune disease. In particular, the damage to the tissue may be caused by or may be a result of osteoarthrosis, periodontal disease, etc.

The glass may be provided to prevent and/or treat damage by the initiation and/or stimulation of tissue repair without incorporation of the bioactive glass into the tissue. Alternatively or in addition, the glass may become incorporated into the tissue, such incorporation of the glass allowing the reconstitution of the tissue. The incorporation of the bioactive glass into the tissue may be permanent or temporary.

In a preferred embodiment, the glass is for use in wound repair by promoting soft tissue regeneration. Shortly after acute tissue injury the microenvironment of a wound is hypoxic. Chronic wounds are the result of insufficient blood vessel formation. The importance of angiogenesis in wound healing is clearly illustrated in the use of angiogenic inhibitors (e.g. endostatin) which delay wound healing and that local VEGF treatment is effective in counteracting this effect. Interestingly, the increased number of ulcers and chronic wounds in elderly patients has been linked to a decreased hypoxia cellular response.

Therefore, in another preferred embodiment, the glass is provided for use in treating ulcers (for example diabetic foot ulcers).

In yet another preferred embodiment, the glass is for promoting bone and hard tissue regeneration and repair. The importance of angiogenesis in bone formation has been recognised for many years. Blood vessels supply the nutrients, growth factors, osteoprogenitor cells and other factors essential for bone formation and bone maintenance in vivo. Increased osteoblast gene expression of the potent angiogenic factor VEGF occurs during fracture repair and the local slow release of VEGF at sites of bone damage in vivo leads to enhanced bone repair and osseointegration. VEGF has also been shown to have a direct role in bone remodelling by stimulating osteoblast differentiation and migration.

For use in promoting bone or hard tissue regeneration and repair the glass is preferably provided in the form of a powder or monolith including a porous scaffold and be used instead of a bone autograft or mixed together with bone autograft material. Bone autografts involve the placement of healthy bone, taken from the patient, into spaces between or around broken bone (fractures) or holds (defects) in the bone. This is advantageous due to the limited amount of bone stock available for transplantation.

In a preferred embodiment the glass is for use in vertebroplasty. The glass may be incorporated into a polymer or cement and injected into the vertebral space by a minimally invasive surgery procedure to prevent osteoporotic fractures and vertebral collapse associated with osteoporosis and resulting in curvature of the spine.

In yet another preferred embodiment, the glass is for treating infection. Advantageously, cellular toxic effects that can be seen with certain anti-microbial agents are avoided by use of a glass of the invention. For example, silver ions can act as an anti-microbial agent, but at certain concentrations can have cellular toxic effects and delay wound healing. The activation of “self-healing” processes (e.g. promoting the recruitment of immune cells and enhancing macrophage phagocytic activity) through the hypoxia pathway is an alternative strategy for enhancing anti-microbial properties.

In a further preferred embodiment, the glass is for use in the enhancement of cytoprotective properties, limiting cell damage and recruiting repair cells (adult stem cells). This may be an important survival mechanism in vivo following ischemic injury (e.g. myocardial infarction). A protective effect on certain cell types is also useful in sustaining the viability of tissue engineered constructs both during transit, during surgery and post-implantation.

In a preferred embodiment, the glass is for use in cartilage repair, reconstruction and regeneration. Cartilage has limited ability to repair itself and consequently tissue engineering is an exciting prospect in cartilage regeneration. The maintenance of chondrocyte phenotype (i.e. their cartilage producing capability) has, however, proven to be difficult in vitro. Tissue scaffolds therefore need to be developed that contain environmental cues that mimic the cartilage ECM and maintain chondrocyte phenotype. In addition to providing a 3-D environment, the development of scaffolds that mimic hypoxia may provide similar environment cues to the low oxygen levels present in native cartilage thus maintaining chondrocyte phenotype and facilitate cartilage TE. Indeed, primary chondrocytes grown in 3D scaffolds in hypoxic environments have been shown to maintain their phenotype. Furthermore, HIF-1α, the master hypoxia sensing transcription factor, is of critical importance in epiphyseal chondrocytes formation, chondrocyte survival, redifferentiation of chondrocytes and differentiation of chondrocyte progenitors.

In a preferred embodiment, the glass is for inducing directed stem cell differentiation. Hypoxia has been demonstrated to be an important regulator in maintaining stem cell plasticity, proliferation and/or differentiation into more specialised cells. The development of 3D scaffolds that regulate the hypoxia response would enable fundamental research and enhance knowledge on maintaining “stemness” and directed differentiation. Hypoxia is known to stimulate the differentiation of angioblasts (circulating endothelial cell precursors) for de novo blood formation, MSCs and nerve cells. For example in response to ischemic injury, previously engrafted, integrated, and quiescent clonal neural stem cells re-enter the cell cycle, migrate preferentially to the site of hypoxia, and differentiate into neurons and oligodendrocytes (the cell types typically destroyed following ischemic brain injury). The recruitment and proliferation of adult stem cells to TE constructs in vivo would greatly enhance tissue repair, development and integration. Preferably, the glass is provided as a scaffold.

In another embodiment, a glass is for use to promote hair growth and/or to increase hair thickness, for example to treat alopecia, hair loss due to aging or as a result of chemotherapy. Angiogenesis, one of the cellular mechanisms stimulated by the hypoxia mimicking effect of transition metal ions released by a glass of the present invention, can modulate hair growth and follicle size. Thus, the glass may be provided in the form of a shampoo.

In another embodiment, the glass of the present invention can be provided as a filler in a degradable polymer e.g. polyester. In particular, the glass can be provided as a filler in a polylactide. A bioactive glass can thus provide a bioactive component for bone screws, fraction fixation plates, porous scaffolds, etc. The use of a glass of the present invention is particularly favoured for use as a filler in a degradable polyester as the bioactive glass prevents autocatalytic degradation which is a feature of polyesters currently known in the art. Autocatalytic degradation occurs as the hydrolysis of an ester results in the formation of an alcohol and an acid. As the hydrolysis of an ester is acid catalysed, the generation of an acid causes a positive feedback situation.

The glass of the present invention may be administered by any convenient method. The glass may be administered topically. Examples of topical application include the administration of a cream, lotion, ointment, powder, gel or paste to the body, for example to the teeth or skin. In particular, the glass can be provided as a toothpaste comprising the glass for administration to the teeth of a patient suffering from dental cavies, periodontal disease, hypersensitive teeth, etc.

The glass may be administered surgically or parenterally. Examples of surgical or parenteral administration would include the administration of the glass into a tissue, by insertion of the device by injection or by a surgical procedure such as implantation, tissue replacement, tissue reconstruction, etc.

The glass can also be administered orally. For oral administration, the composition can be formulated as a liquid or solid, for example solutions, syrups, suspensions or emulsions, tablet, capsules and lozenges. Administration of the glass by oral or parental administration may provide the glass directly at its required site of action. Alternatively, the glass can be delivered to its site of action, for example by using the systemic circulation. The glass can be administrated orally, for example to a patient requiring the prevention and/or treatment of damage to the alimentary canal.

The compositions can be used in the form of particles, three dimensional scaffolds, monoliths, coatings and/or fibres, among other possible forms and can be used, for example, for stimulating angiogenesis (new blood vessel formation), stimulating lymphangiogenesis (new lymphatic vessel formation), for antimicrobial purposes and directing the proliferation of cells and differentiation of progenitor/stem cells. These properties can be used, for example, enhancing wound healing, fighting infection, stimulating bone growth, stimulating hair follicle growth, regenerating cartilage tissue and other therapeutic or cosmetic purposes. The hypoxia pathway regulating glasses can be incorporated into or onto implanted materials, tissue regeneration constructs and wound healing devices, such as tissue scaffolds, sutures, prosthetic implants, polymer matrices, fibrin gels, hydrogels, plasters, wound dressings, creams, shampoo, aerosols and the like. The hypoxia stimulating glass compositions can also be used in devices used for in vitro and ex vitro cell culture.

All preferred features of each of the aspects of the invention apply to all other aspects mutatis mutandis. It will also be appreciated that the various embodiments of the invention may be present in combination.

The invention may be put into practice in various ways and a number of specific embodiments will be described by way of example to illustrate the invention, with reference to the accompanying figures in which:

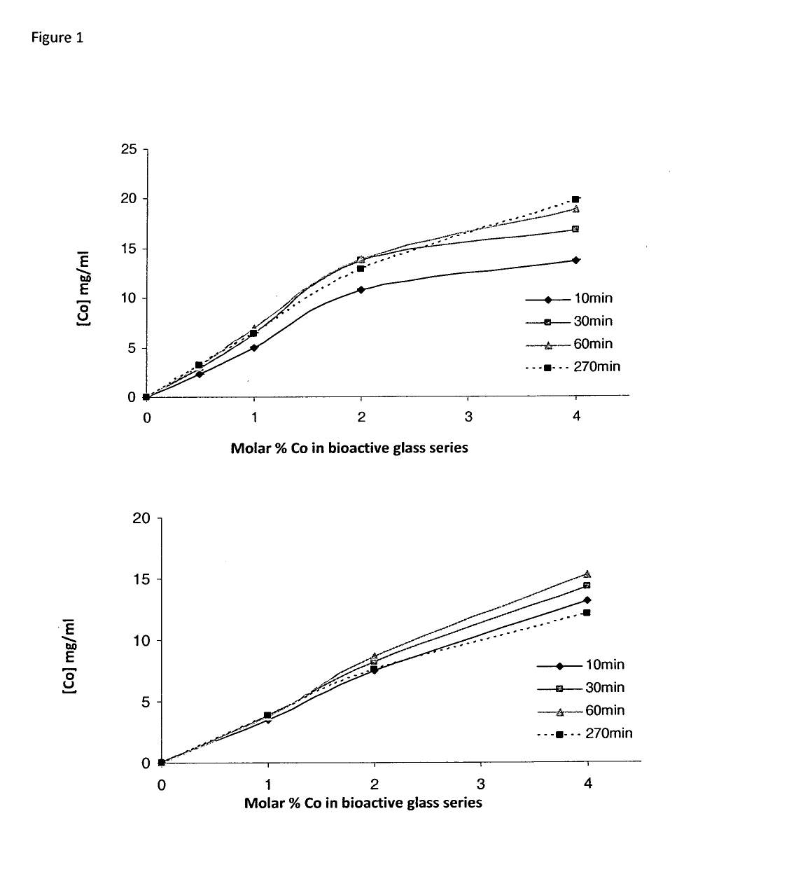

FIG. 1 shows the results of inductively coupled plasma (ICP) analysis of ion release from a series of cobalt-containing glasses: a) (top trace) glass examples 1-4; b) (lower trace) glass examples 5-7.

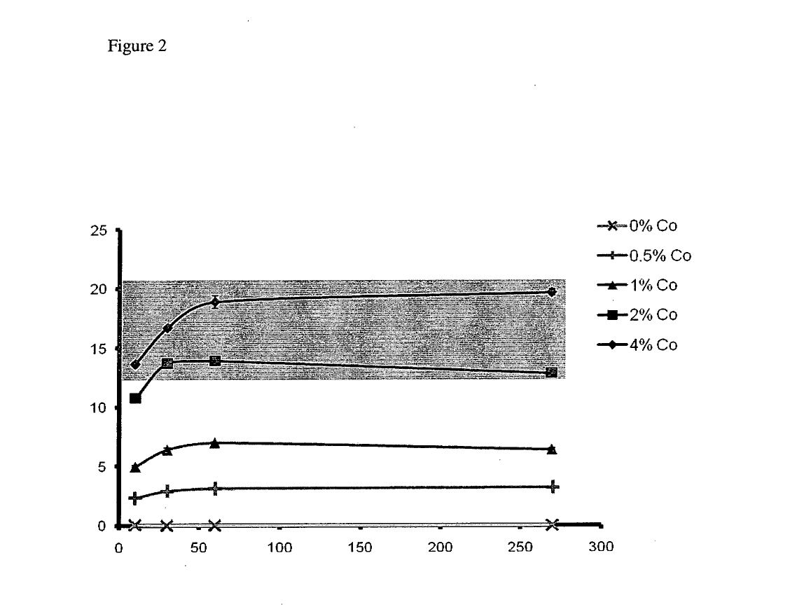

FIG. 2 shows controlled, chemical composition dependant, cobalt ion release over time with inductively coupled plasma (ICP) analysis from a series of cobalt-containing glasses in Tris buffer (examples 1-4).

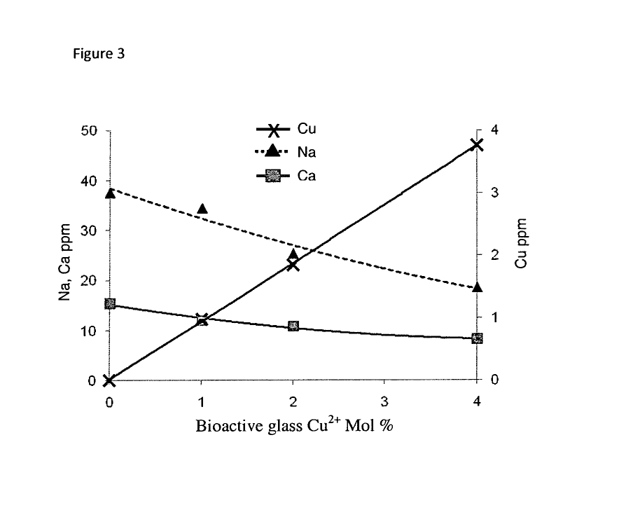

FIG. 3 shows the results of ICP analysis of Cu, Na and Ca released from a series of copper-containing glasses after 30 minutes incubation in Tris buffer (examples 1-4, with Co substituted by Cu).

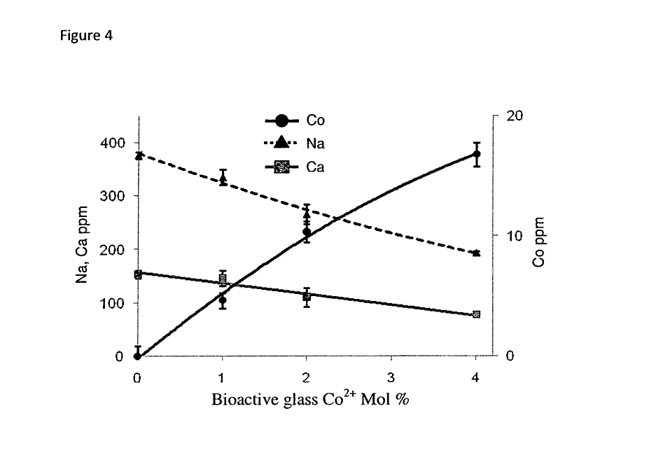

FIG. 4 shows the results of ICP analysis of Co, Na and Ca released from a series of cobalt-containing glasses after 30 minutes incubation in Tris buffer (examples 1-4).

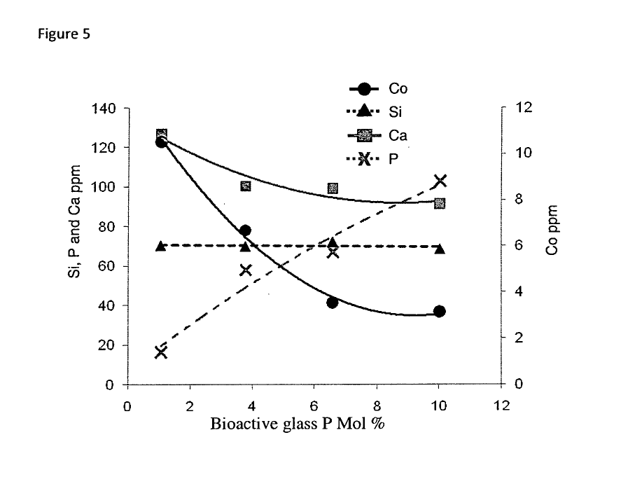

FIG. 5 shows the results of ICP analysis of P, Co, Si and Ca released, after 30 minutes incubation in Tris buffer, from a series of cobalt-containing glasses which have an increasing Mol % concentration of P (examples 13-16).

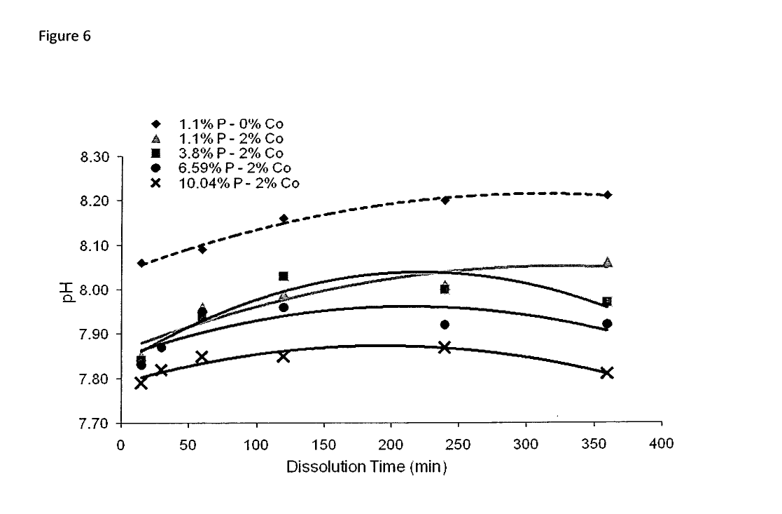

FIG. 6 shows the pH change caused by various hypoxia mimetic glasses (examples 3 and 13-15) in distilled water.

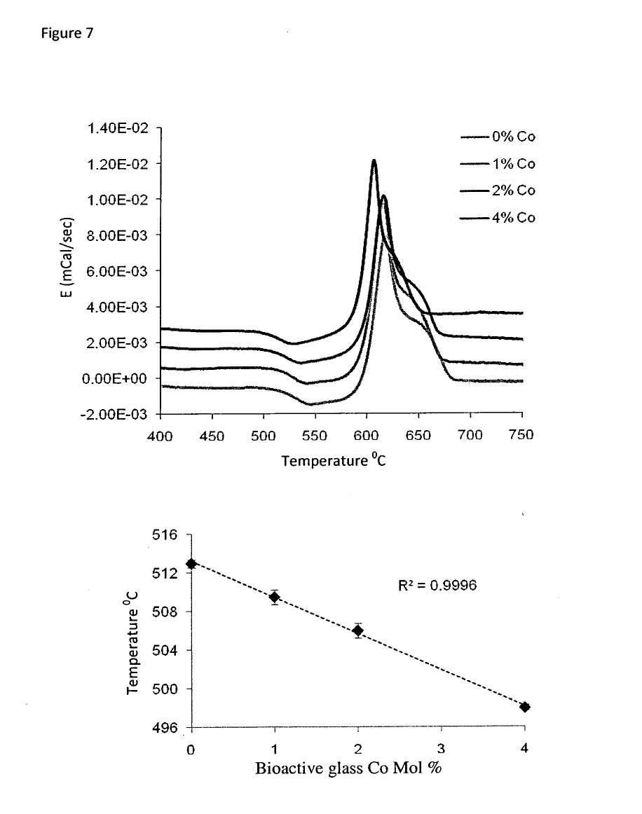

FIG. 7 shows Differential Scanning calorimetry (DSC) traces of certain cobalt-containing glass compositions (examples 1-4).

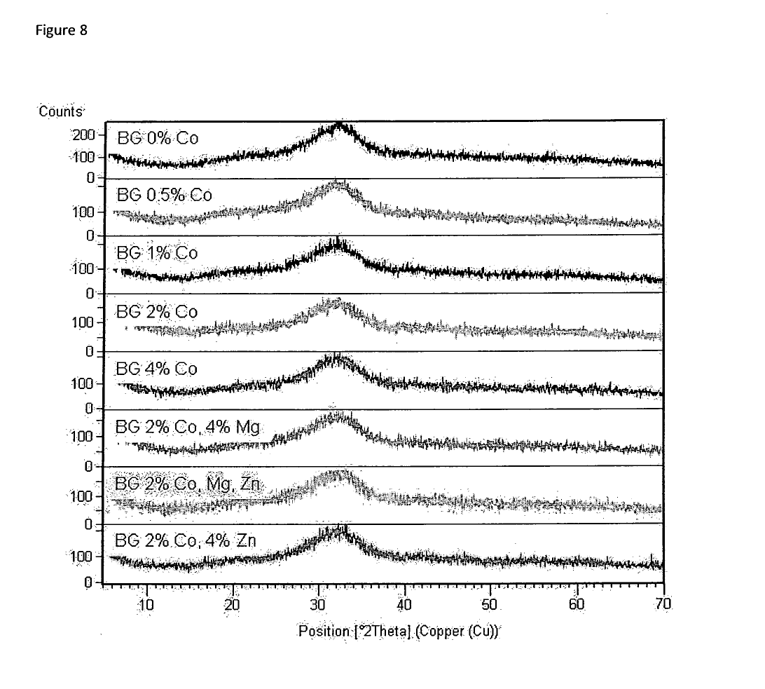

FIG. 8 shows XRD traces of examples of hypoxia mimetic glasses (compositional examples 1-4, 34, 44 and 45).

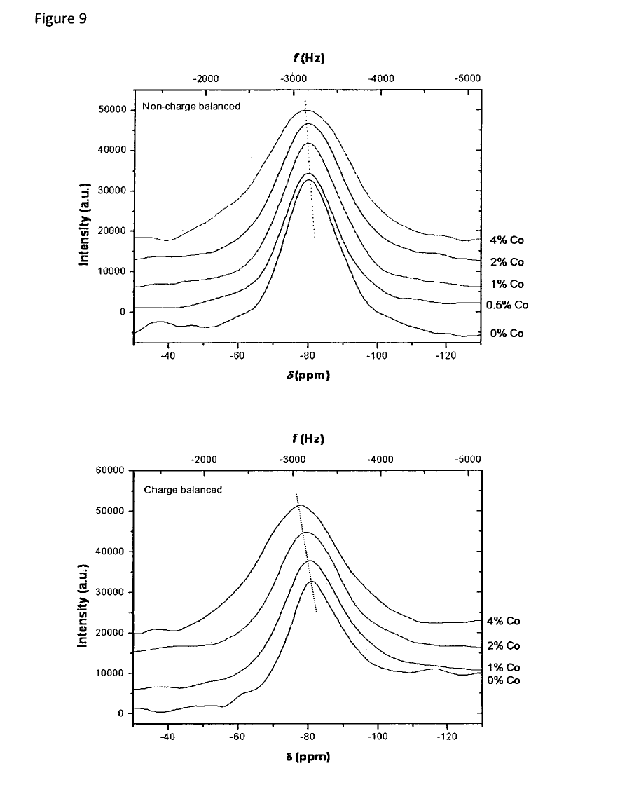

FIG. 9 shows ²⁹Si MAS-NMR (Magic angle spinning-nuclear magnetic resonance) of cobalt-containing glasses. (examples 1-4 and 5-7)

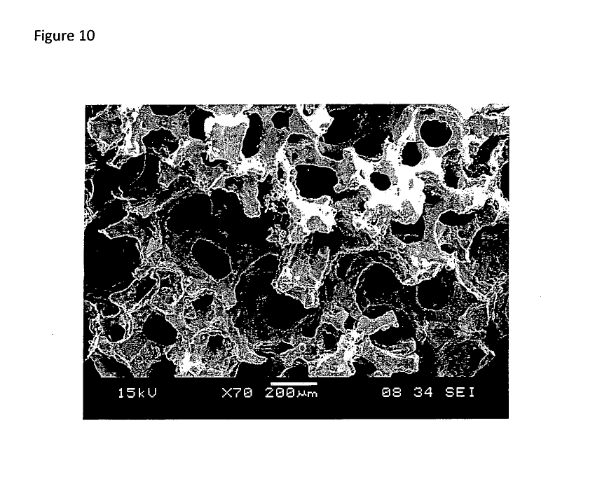

FIG. 10 shows a SEM image of a porous hypoxia bioactive glass scaffold (example 24) produced using a gel cast foaming method.

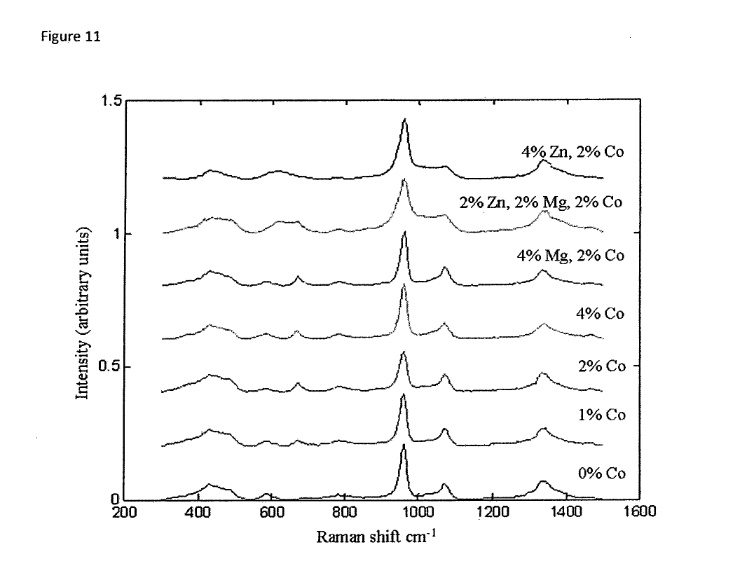

FIG. 11 shows Raman spectra of hypoxia mimetic bioactive glasses of various compositions incubated in simulated body fluid (SBF) for 3 weeks. Apatite (PO₄³⁻ 960 cm⁻¹) formation was present on all these particular hypoxia mimetic bioactive glass compositions (examples 1-4 and 34, 44 and 45).

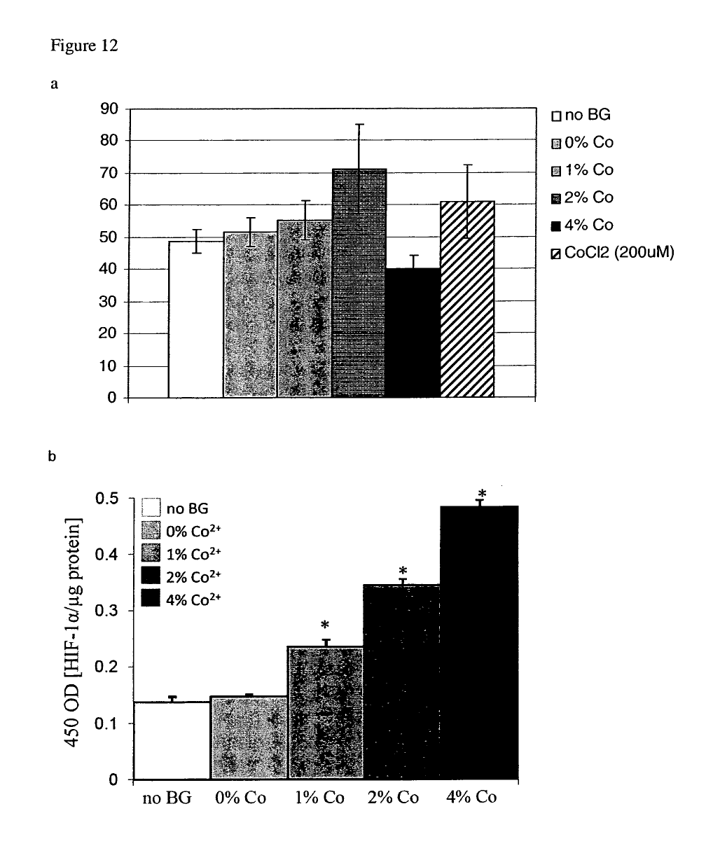

FIG. 12a shows transcription factor HIF-1α expression (vertical axis being [HIF-1α]/[Cyt.C]) and FIG. 12b shows transcription factor HIF-1α stabilization, both observed in cell culture experiments with media conditioned with glasses (BG) containing different concentrations of cobalt ions (0-4 mol %, examples 1-4) for 48 hours.

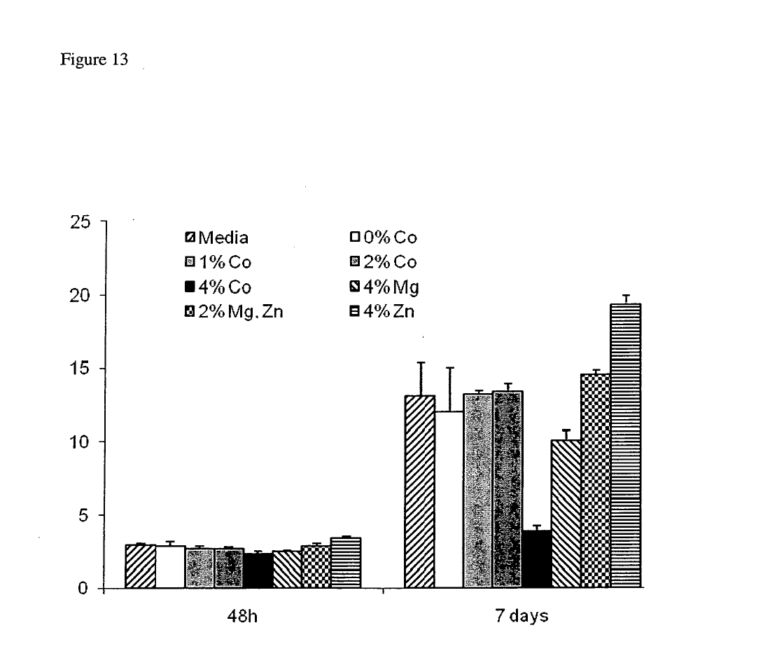

FIG. 13 shows the viability (total DNA) of osteoblasts cultures for 48 hrs and 7 days in media conditioned with various hypoxia mimetic bioactive glasses (examples 1-4, 34, 44 and 45)—for glasses where Zn or Mg content is indicated, 2% CoO is also present.

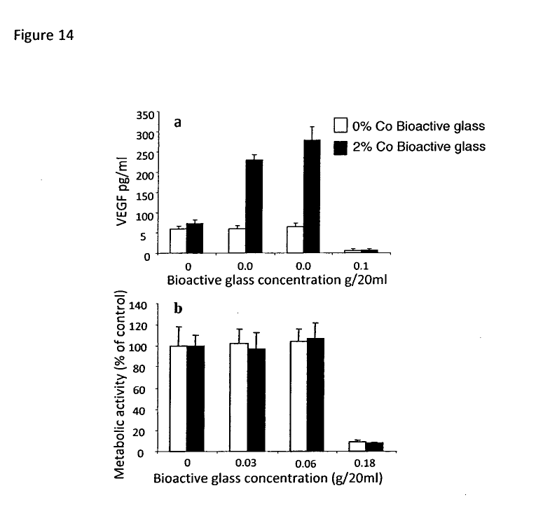

FIG. 14 shows (a) the VEGF expression seen in osteoblast (MG63) cell cultures exposed to glasses with cobalt (example 3) and without cobalt at various concentrations for 24 hours; and (b) the metabolic activity observed in these cultures.

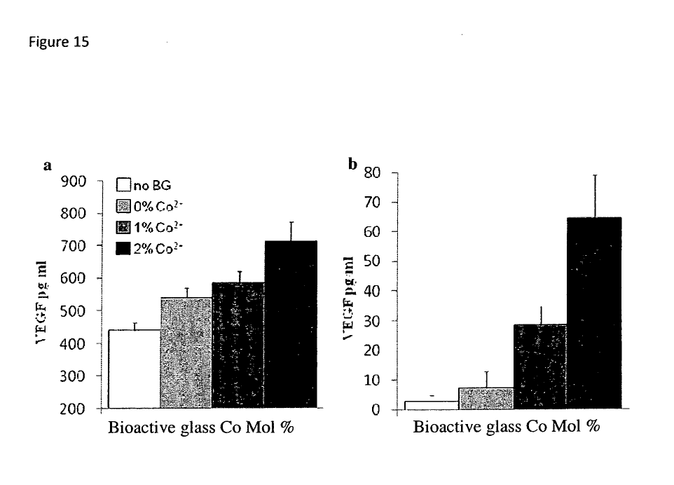

FIG. 15 shows the VEGF expression seen in (a) endothelial cell (HMEC-1) and (b) osteoblast cells (SaOS-2) cultured for 24 hours in media conditioned with various hypoxia mimetic bioactive glasses (0-4 mol Co %, examples 1-4).

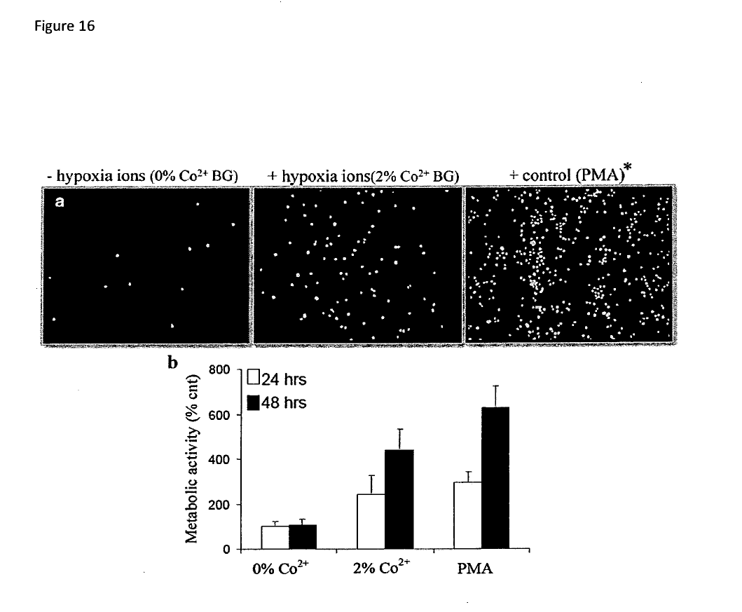

FIG. 16 shows the differentiation of non-adherent monocytes to adherent macrophage-like cells when exposed to hypoxia mimetic glasses (2% Co example 3).

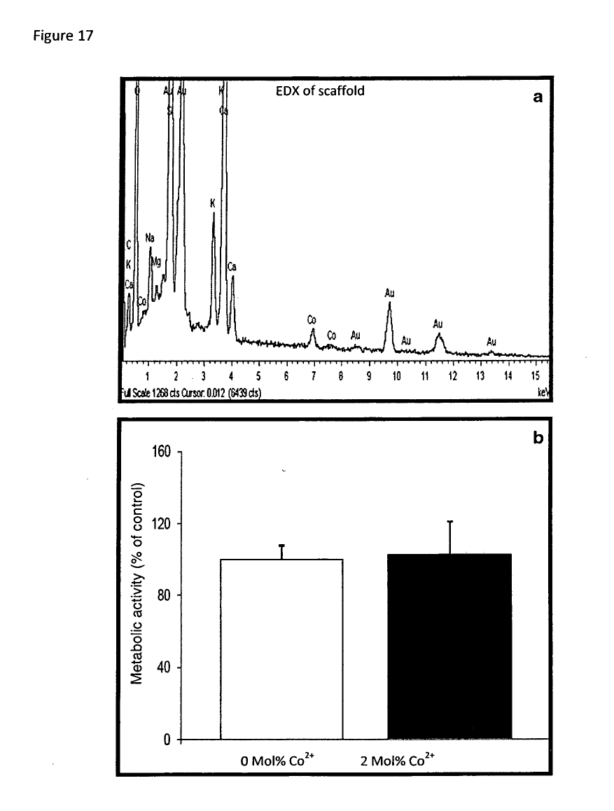

FIG. 17a shows EDX image of a porous hypoxia bioactive glass scaffold (example 27) produced using a gel cast foaming method after 4 days endothelial (HMEC-1) cell culture. FIG. 17b shows the relative metabolic activity of HMEC-1 cells cultured on the gel-cast scaffold after 4 days culture (normalised to control glass without hypoxia ions).

The meanings of terms used herein are explained below, and the invention will now be further illustrated with reference to one or more of the following non-limiting examples.

In the context of this invention, a glass is a bioactive glass if, when implanted into living tissue, it induces formation of an interfacial bond between the glass and the surrounding tissue. An in vitro index of bioactivity is provided by the rate of development of a hydroxycarbonated apatite (HCA) layer on the surface of a glass. In certain preferred embodiments a bioactive glass is one where, on exposure of the glass to simulated body fluid (SBF), deposition of a crystalline HCA layer occurs within 3 days, more preferably within 24 hours. Deposition of a HCA layer on exposure to SBF (as described in Kokubo T., J. Biomed. Mater. Res. 1990; 24; 721-735) is a recognised test of bioactivity.

### Exemplary Glass Compositions in Mole Percent

Certain glass compositions of the invention are set out in the table below. Analysis which has been carried out on a number of these glasses is described in the following examples.

These glasses can be produced using melt-derived glass production techniques, involving mixing and blending the appropriate oxides (or sources of oxides, such as carbonates), melting and homogenising (for example by oxygen bubbling) the mixture at a temperature of approximately 1250-1500° C. and cooling the mixture, for example by casting the molten mixture into a suitable liquid such as water, to produce a glass frit. Standard calculations based on molecular weights and the mol % compositions as set out in the table below can be used to determine the mass of each component required in the glass melt mixture. Thus, the appropriate amount of the various oxides (or oxide sources) to use in the melt mixture can be calculated based on the values set out in the table below. By means of example, glass example 26 can be prepared by mixing 46.53% SiO₂, 27.27% CaCO₃, 6.47% Na₂CO₃, 6.47% K₂CO₃, 2.94% ZnO, 2.94% MgO, 1.96% CoCO₂, 2.94% SrCO₃ and 1.05% P₂O₅.

| | | | | | | | | | | | | CoO, CuO (or |
|---|---|---|---|---|---|---|---|---|---|---|---|---|
| | | | | | | | | | | | | other hypoxia |
| Examples | Preferred Application | SiO₂ | P₂O₅ | B₂O₃ | CaO | SrO | Na₂O | K₂O | ZnO | MgO | CaF₂ | ion eg NiO) |
| 1 | Hypoxia glass (1) | 49.46 | 1.07 | | 22.58 | | 26.38 | | | | | 0.50 |
| 2 | Hypoxia glass (2) | 49.49 | 1.07 | | 22.08 | | 26.38 | | | | | 1.00 |
| 3 | Hypoxia glass (3) | 49.46 | 1.07 | | 21.08 | | 26.38 | | | | | 2.00 |
| 4 | Hypoxia glass (4) | 49.46 | 1.07 | | 19.08 | | 26.38 | | | | | 4.00 |
| 5 | Charged Balanced (1) | 48.94 | 1.08 | | 24.32 | | 26.64 | | | | | 1.01 |
| 6 | Charged Balanced (2) | 48.40 | 1.09 | | 25.58 | | 26.90 | | | | | 2.04 |
| 7 | Charged Balanced (3) | 47.27 | 1.11 | | 28.16 | | 27.43 | | | | | 4.16 |
| 8 | Charged Balanced (4) | 47.98 | 1.06 | | 23.84 | | 26.12 | | | | | 0.99 |
| 9 | Charged Balanced (5) | 46.53 | 1.05 | | 24.59 | | 25.87 | | | | | 1.96 |
| 10 | Charged Balanced (6) | 43.72 | 1.03 | | 26.04 | | 25.37 | | | | | 3.85 |
| 11 | Bone (1) | 49.46 | 1.07 | | 15.81 | 5.27 | 26.38 | | | | | 2.00 |
| 12 | Bone (2) | 49.46 | 1.07 | | 10.54 | 10.54 | 26.38 | | | | | 2.00 |
| 13 | Bone (3) | 49.46 | 1.07 | | 0.00 | 21.08 | 26.38 | | | | | 2.00 |
| 14 | High Phosphate - pH stable | 48.84 | 3.79 | | 22.44 | | 27.93 | | | | | 2.00 |
| | glass (1) | | | | | | | | | | | |
| 15 | High Phosphate - pH stable | 38.06 | 6.59 | | 25.83 | | 27.52 | | | | | 2.00 |
| | glass (2) | | | | | | | | | | | |
| 16 | High Phosphate - pH stable | 30.93 | 10.04 | | 27.55 | | 29.49 | | | | | 2.00 |
| | glass (3) | | | | | | | | | | | |
| 17 | Bone + hypoxia + Sr | 49.46 | 1.07 | | 17.81 | 5.27 | 24.38 | | | | | 2.00 |
| 18 | Bone + hypoxia + Sr + Zn | 49.46 | 1.07 | | 15.81 | 5.27 | 24.38 | | 2.00 | | | 2.00 |
| 19 | Bone + hypoxia + Sr + Zn + K | 49.46 | 1.07 | | 15.81 | 5.27 | 12.19 | 12.19 | 2.00 | | | 2.00 |
| 20 | Bone + hypoxia + Sr + Zn + K + | 49.46 | 1.07 | | 13.81 | 5.27 | 12.19 | 12.19 | 2.00 | 2.00 | | 2.00 |
| | Mg | | | | | | | | | | | |
| Examples | Application | SiO₂ | P₂O₅ | B₂O₃ | CaO | SrO | Na₂O | K₂O | ZnO | MgO | CaF₂ | Hypoxia ion |
| 21 | Bone + hypoxia + Sr + Zn + K + | 47.46 | 1.07 | 2 | 13.81 | 5.27 | 12.19 | 12.19 | 2.00 | 2.00 | | 2.00 |
| | Mg + B | | | | | | | | | | | |
| 22 | Periodontal treatment (1) | 49.46 | 1.07 | | 17.08 | | 6.60 | 13.18 | 2.0 | 2.0 | 6.60 | 2.00 |
| 23 | Periodontal treatment (2) + | 49.46 | 1.07 | | 12.08 | 5 | 6.60 | 13.18 | 2.0 | 2.0 | 6.60 | 2.00 |
| | Sr | | | | | | | | | | | |
| 24 | Glass for Porous Sintered | 49.46 | 1.07 | | 25.27 | 3.00 | 6.6 | 6.60 | 3.00 | 3.00 | | 2.00 |
| | Scaffold for bone (1) | | | | | | | | | | | |
| 25 | Porous Sintered Scaffold | 47.46 | 1.07 | 2.0 | 11.64 | 10.00 | 6.60 | 6.60 | 3.00 | 3.00 | 8.64 | 2.00 |
| | for bone (2) | | | | | | | | | | | |
| 26 | Porous Sintered Scaffold | 46.53 | 1.05 | | 27.27 | 2.94 | 6.47 | 6.47 | 2.94 | 2.94 | | 1.96 |
| | for bone (3) | | | | | | | | | | | |
| 27 | Porous Sintered Scaffold | 46.48 | 1.05 | | 37.58 | | 6.46 | 6.46 | | | | 1.96 |
| | for bone (4) | | | | | | | | | | | |
| 28 | Filler for Composites | 49.46 | 1.07 | | 40.86 | | 6.6 | | | | | 2.00 |
| 29 | Filler for Composites + Sr | 49.46 | 1.07 | | 21.43 | 19.43 | 6.6 | | | | | 2.00 |
| 30 | Filler for non bone | 49.46 | 1.07 | | 30.86 | | 6.6 | | 5 | 5 | | 2.00 |
| | Composites | | | | | | | | | | | |
| 31 | Soft tissue (high Si) | 50.46 | 1.05 | | 18.71 | | 25.87 | | | | | 3.92 |
| 32 | Soft tissue (high Si) | 51.41 | 1.03 | | 18.35 | | 25.37 | | | | | 3.85 |
| 33 | Soft tissue (high Si) | 52.33 | 1.01 | | 18.00 | | 24.89 | | | | | 3.77 |
| 34 | Soft tissue (Zn) | 49.46 | 1.07 | | 17.08 | | 26.38 | | 4 | | | 2.00 |
| 35 | Soft tissue (Zn) | 49.46 | 1.07 | | 16.58 | | 26.38 | | 6 | | | 0.50 |
| 36 | Soft tissue (Zn) | 49.46 | 1.07 | | 15.08 | | 26.38 | | 6 | | | 2.00 |
| 37 | Soft tissue (Zn) | 49.46 | 1.07 | | 12.08 | | 26.38 | | 6 | | | 5.00 |
| 38 | Soft tissue | 49.46 | 1.07 | | 16.58 | | 26.38 | | 6 | | | 0.25 Co + .25 |
| | | | | | | | | | | | | Cu |
| 39 | Soft tissue (Zn) | 49.46 | 1.07 | | 15.08 | | 26.38 | | 6 | | | 1 Co + 1 Cu |
| 40 | Soft tissue (Zn) | 49.46 | 1.07 | | 12.08 | | 26.38 | | 6 | | | 2.5 Co + 2.5 Cu |
| 41 | Soft tissue (Zn + Mg) | 49.46 | 1.07 | | 15.08 | | 26.38 | | 3 | 3 | | 2.00 |
| 42 | Soft tissue (Zn + Mg) | 49.46 | 1.07 | | 13.08 | 2.00 | 26.38 | | 3 | 3 | | 2.00 |
| 43 | Soft tissue (Mg) | 49.46 | 1.07 | | 13.08 | 2.00 | 26.38 | | | 6 | | 2.00 |
| 44 | Soft tissue (Mg) | 49.46 | 1.07 | | 17.08 | | 26.38 | | | 4 | | 2.00 |
| 45 | Soft tissue (Zn + Mg) | 49.46 | 1.07 | | 14.08 | | 26.38 | | 2 | 2 | | 2.00 |
| 46 | Soft tissue | 57.46 | 1.07 | 2 | 15.08 | | 16.38 | | 6 | | | 2.00 |
| | applications | | | | | | | | | | | |
| 47 | Soft tissue | 61.46 | 1.07 | 2 | 13.08 | 2.00 | 10.38 | 2.00 | 3 | 3 | | 2.00 |
| | applications | | | | | | | | | | | |
| 48 | Soft tissue | 49.46 | 1.07 | | | | 26.38 | | | 21.08 | | 2.00 |
| | applications | | | | | | | | | | | |
| 49 | Bioactive glass | 49.46 | 1.07 | | 18.08 | | 26.38 | | 3.00 | | | 4.00 |
| | Shampoo for alopecia | | | | | | | | | | | |
| | (1) | | | | | | | | | | | |
| 50 | Bioactive glass | 49.46 | 1.07 | | 20.58 | | 26.38 | | 6.00 | | | 0.5 Co + 2.0 Cu |
| | Shampoo for alopecia | | | | | | | | | | | |
| | (2) | | | | | | | | | | | |

### Supporting Experimental Data

Hypoxia mimicking ions were successfully incorporated into the silica network of resorbable glasses of the invention. Glass compositions of the invention as shown in the table above have been characterised by ICP, pH, DSC, X-ray diffraction, NMR and SEM analysis. The composition and manufacture of the glasses was successfully manipulated to allow the controlled release of hypoxia mimetics at physiological relevant ranges. Furthermore, in addition to the control release of hypoxia mimetics the chemical compositions of the glasses were developed in such a way to control the release of other ions, which are important to determining biological response (e.g. apatite formation or cell behaviour). The development, composition and characterization of these glasses is described in detail herein.

The dissolution products from the hypoxia mimetic glasses (the hypoxia mimicking ions) stabilized the transcription factor hypoxia-inducible factor-1α (HIF-1α) in normal oxygen pressure environments (FIG. 12), without toxicity (FIGS. 13 & 14), induced the transcription of HIF-targeted genes such as VEGF (FIGS. 14 & 15) and caused cell differentiation (FIG. 16). The cellular response of various cell types to the hypoxia mimetic glasses of the invention were studied, including human osteoblast-like osteosarcoma cells (SaOS-2 and MG63), human umbilical cord vascular endothelial cells (HUVECs) and monocyte cells (U937). For example, human osteoblast-like osteosarcoma cells (SaOS-2) have been cultured in RPMI medium containing 10% (v/v) FBS, L-Glutamine (2 mM), 1% (v/v) antibiotic and seeded (50,000/cm²) on 48 well plates with either hypoxia ion containing glass conditioned media or control glass condition media. Cells have also been successfully grown directly onto 3-D scaffolds formed from glasses of the invention produced using a gel cast foaming method (glass 27, FIG. 17).

Experimental work has therefore confirmed the feasibility of creating bioactive glass networks containing hypoxia ions (eg Co ions), control over the hypoxia ion release rates and generating desired cellular responses without cytotoxicity i.e. the activation of the HIF-1α pathway (FIG. 12) and associated regenerative responses, e.g. increased expression of the potent angiogenic factor VEGF (FIGS. 14 and 15). For example, in some experiments, cobalt bioactive glass conditioned media has been shown to induce a concentration dependant 6-fold increase in VEGF production (p<0.01) after just 24 hrs without toxicity.

FIGS. 1 and 2 shows inductively coupled plasma (ICP) analysis of ion release over time. The composition of the glass determines (hypoxia mimetic) cobalt ion release rates, thereby allowing predictive hypoxia mimetic release profiles. The release profile is dependent upon the manufacture and glass chemical composition ranges as determined by this invention. The composition series of the hypoxia regulating bioactive glasses in FIG. 1a and 2 are listed as glass examples 1-4, whilst the composition of a charged balanced series of the glasses in FIG. 1b are listed as glass examples 5-7. The control glass (0% Co) in all experiments described herein consisted of SiO₂ (49.46 mol %), P₂O₅ (1.07 mol %), CaO (23.08 mol %) and Na₂O (26.38 mol %). A linear correlation (0.99 regression) exists between molar % of cobalt within the glass and release when compositions were constructed to maintain the same network connectivity (NC=2.13 (FIG. 1b)), whilst a non-linear polynomial correlation exists when network connectivity is increased (FIG. 1a). The composition of these hypoxia regulating bioactive glasses released Co²⁺ within the known physiologically active range (48 μM-240 μM). As these glasses are bioactive (FIG. 13) they are useful, among other things, in applications requiring bone tissue regeneration (by promoting blood vessel formation together with apatite formation e.g. as bone fillers or in bone tissue engineering).

FIGS. 3 and 4 show the release of hypoxia mimetics (Cu and Co respectively) is determined by the chemical composition of the glasses. Increasing the molar % of the hypoxia mimetics within the glass network also modifies the network of the glass, changing the ion release kinetics of other glass ions (e.g. Na) in a predictive manner.

FIG. 5 shows that increasing the molar % of phosphorous within the glass network decreases the release of hypoxia mimetics (Co²⁺), whilst maintaining Si (glass network former) release profiles. The glass mol % P, in these particular chemical compositions, can thereby be used to control hypoxia mimetic release. Hypoxia mimetic glasses with higher phosphate mol % can be used to reduce the pH change observed with lower mol % P concentration (1.04% P) bioactive glasses (FIG. 6) and those previously shown by Bioglass®. Reducing the number of available H⁺ ions by increasing P molar % can be used to manipulate bioactivity (apatite forming ability) and minimise any potential cell toxicity from basic pH environments. These glasses could therefore be used in applications where minimising pH change is important (e.g. cell culture systems, shampoo).

FIG. 7 shows Differential Scanning calorimetry (DSC) traces. An increasing concentration of cobalt within certain glass compositions unexpectedly reduced the transition temperature of the glasses. The role of cobalt as a network former or modifier within the glass depends upon the composition of the glass and concentration of cobalt. The reduction in the glass transition temperature indicates CoO is going into the glass structure.

FIG. 8 shows XRD traces of glasses of the invention. The amorphous halo and lack of sharp peaks indicates that the material was still amorphous after sintering. There is no evidence of crystalline Co phases and Co is incorporated into the glass structure.

FIG. 9 shows ²⁹Si MAS-NMR (Magic angle spinning-nuclear magnetic resonance) of glasses of the invention (hypoxia bioactive glasses). ²⁹Si MAS-NMR revealed that whilst the line width of the samples increased with increasing cobalt concentration (a paramagnetic effect of cobalt), the glass structure did not change and was therefore able to maintain glass properties. FIG. 9a shows data for glass examples 1-4, whilst FIG. 9b shows data for glasses from the charged balanced series (examples 5-7).

FIG. 10 shows a SEM image of a porous hypoxia bioactive glass scaffold produced using a gel cast foaming technique involving the foaming of a glass particulate slurry containing glass example 27 and in situ polymerisation of gelling agents. The gelled foam can then be poured into a mould immediately prior to gelation and heat treated to remove the polymer and sinter the glass particles. The controlled release of hypoxia mimetic ions released from the scaffold will promote angiogenesis, a vital component of new functional bone growth and survival.

FIG. 11 shows that certain hypoxia mimetic bioactive glass compositions form apatite (PO₄³⁻ 960 cm⁻¹) when incubated with SBF for 3 weeks. The amount of apatite formed (area of 960 cm⁻¹), full width half maximum of the apatite peak (960 cm⁻¹) and the carbonate (CO₃²⁺) to phosphate (PO₄³⁻) ratio varied dependent upon chemical composition and SBF incubation period. This allows the production of hypoxia mimetic glasses with specific bioactivity for specific applications.

### Cell Culture Experiments

The ability of the dissolution products from the hypoxia mimetic glasses to stabilize expression of HIF-1α and promote tissue regenerative responses, whilst minimizing cytotoxicity was determined by HIF-1α transcription factor assays (R&D systems and TransAM™ Active Motif) immunofluorescence, VEGF ELISA, LDH (CytoTox 96® Non-Radioactive Cytotoxicity Assay), total DNA and MTT respectively. The cellular response of various cell types to the dissolution products from the glasses of the invention were studied, including human osteoblast-like osteosarcoma cells (SaOS-2 and MG63), human microvascular endothelail cell line-1 (HMEC-1) and human monocyte cell lines (U937).

Human microvascular endothelail cell line-1 (HMEC-1) were cultured in 1% gelatin (bovine skin, Sigma) coated 75 cm³ flasks with endothelial basal medium, MCDB-131 supplemented with 10% FBS (Sigma), 10 ng/ml epidermal growth factor (EGF Sigma), 1 ug/ml hydrocortisone (Sigma), 1% antibiotic-antimycotic and 5% L-glutamine (Sigma). SaOS-2 and U937 cells were cultured in RPMI medium containing 10% (v/v) FBS and L-Glutamine (2 mM). The human bone-like osteosarcoma cell line MG-63 (ECCAC) was cultured in DMEM supplemented with 10% foetal bovine serum, 1% non-essential amino acids and L-Glutamine (2 mM). The glass (BG) conditioned media was generated by incubation of glass particles (<45 μm diameter) of various compositions, in 20 ml of the desired media (DMEM, RPMI, MCDB-131 for 4 hours at 37° C., prior to centrifugation and filtration to remove particles.

FIG. 12a shows a Co-glass concentration dependant increase in the expression of HIF-1α. Expression of Hif-1α a was normalised to cytochrome c (Cyt.C). CoCl₂ was used as a positive control for HIF-1α expression. FIG. 12b shows that hypoxia mimetic glasses stabilise the expression of HIF-1α(P<0.05) in a concentration dependant manner (mol % of hypoxia mimetic) and thereby allowing its translocation into the nucleus and hypoxia related gene expression. No significant increase in HIF-1α stabilisation was observed in conditioned media from control glasses without hypoxia mimetics (0 mol % Co).

FIG. 13, shows the cell viability (amount of DNA) of osteoblasts (SaOS-2) cultured for 48 hours or 7 days in conditioned media from hypoxia mimetic glasses of various compositions. The various hypoxia mimetic glass compositions do not affect cell viability over this culture period in all glass compositions apart from the hypoxia mimetics with 4 mol % Co (example 4) cultured for 7 days. This shows that the hypoxia glass mol % concentration of cobalt and the release profile is important in minimising cytotoxicity.

FIG. 14 shows the results of cell culture experiments with media conditioned with hypoxia glasses (example 3) and control glass (0% Co, 49.46 mol % Si, 1.07 mol % P₂O₅, 23.08 mol % CaO and 26.38 mol % Na₂O) at various concentrations. A dramatic cobalt-bioactive glass concentration dependant increase in the expression of the potent angiogenic factor, VEGF, by osteoblasts (MG63) after just 24 hours culture (a). Parallel experiments revealed no hypoxia bioactive glass specific toxicity (compared to bioactive glass without hypoxia stimulating Co ions) (b). The cobalt bioactive glass conditioned media induced a concentration dependant 6-fold increase in VEGF production (p<0.01) after just 24 hrs without toxicity (FIG. 12).

FIG. 15 shows dramatic and concentration dependant increases in the expression of the potent angiogenic factor VEGF by monocytes (U937s) and endothelial cells (HMEC-1) after 48 hour culture in hypoxia bioactive glass conditioned media of increasing Co mol % (glass examples 1-4). The amount of VEGF expressed is specific to cell type and hypoxia glass chemical composition.

FIG. 16 shows hypoxia glass induced monocyte differentiation. In this example, the number of differentiated adherent monocyte-like cells (U937s) as opposed to non-adherent undiffererentiated U937s was measured by fluorescent DAPI staining (a) and metabolic activity (b). A significantly increase U937 differentiation occurred in hypoxia glass (BG) conditioned media (0.03 g/20 ml of compositional example 3 was incubated for 3 hours) compared to control glass (3 g/20 ml of 0% Co²⁺ control glass incubated for 3 hours). Hypoxia glass controlled monocyte differentiation was, however, less than PMA (phorbol-myristate acetate) induced differentiation. The recruitment of monocytes and their differentiation into macrophages is an important part of wound healing and fighting potential microbial infection. PMA is a positive control to demonstrate that cells can differentiate.

### Production of a Porous Glass Scaffold

A porous glass scaffold was produced from glass example 27 using a gel cast foaming method. The scaffold was then immersed in MCDB-131 for 24 hours prior endothelial cell seeding (20,000 cells/ml, HMEC-1 cells) for 4 days. SEM imaging of the scaffold showed a semi-confluent endothelial cell layer growing on the scaffold after 4 days cell culture. A well-spread, inter-connective morphology of endothelial cells was observed, suggesting that the endothelial cells were viable and that the scaffold is non-cytotoxic. Energy dispersive X-Ray (EDX) analysis revealed that hypoxia mimetic (Co) was still present in the glass network after 4 days cell culture.

It should be appreciated that various changes and modifications to the presently preferred embodiments described herein will be apparent to those skilled in the art. Such changes and modifications, which can be made without departing from the spirit and scope of the invention, fall within the scope of the invention.

1. A glass comprising:
30-62% SiO₂;
0.5-15% P₂O₅;
a combined content of CaO and SrO of 12-45%;
a combined content of Na₂O and K₂O of 6-30%; and
0.1-10% of a source of hypoxia mimicking ions,
wherein the hypoxia mimicking ions are selected from one or more of Co, Cu, Mn, Ni and Fe ions.

2. The glass of claim 1, wherein the glass comprises 45-62%, SiO₂, 0.5-1.5% P₂O₅ and a combined content of Na₂O and K₂O of 6-28% and/or wherein the glass comprises MgO at 0-12%, ZnO at 0-10%, B₂O₃ at 0-15% and a source of fluorine at 0-10%.

3. The glass of claim 1, wherein the glass comprises an amorphous glass network and the hypoxia mimicking ions are integrated into the amorphous glass network.

4. The glass of claim 1, wherein the hypoxia mimicking ions are Co or Cu.

5. The glass of claim 1, wherein the source of hypoxia mimicking ions is present at 0.5-5 mol %.

6. The glass of claim 1, wherein the glass comprises: 47-50% SiO₂; 0.5-15% P₂O₅; 0-2% B₂O₃; a combined molar percentage of CaO and SrO of 18-25%; a combined molar percentage of Na₂O and K₂O of 24-27%; 0-2% ZnO; 0-2% MgO and 0.1-10% of a source of hypoxia mimicking ions.

7. (canceled)

8. The glass of claim 1, wherein the glass comprises: 49-50% SiO₂; 0.5-15% P₂O₅; a combined molar percentage of CaO and SrO of 16-18%; a combined molar percentage of Na₂O and K₂O of 18-20%; 1-3% ZnO; 1-3% MgO and 0.1-5% of a source of hypoxia mimicking ions.

9. (canceled)

10. The glass of claim 1, wherein the glass comprises: 46-50% SiO₂; 0.5-15% P₂O₅; 0-2% B₂O₃; a combined molar percentage of CaO and SrO of 20-29%; a combined molar percentage of Na₂O and K₂O of 12-14%; 1-10% a source of fluorine; 2-4% ZnO; 2-4% MgO; 0-9% CaF₂ and 0.1-5% of a source of hypoxia mimicking ions.

11. (canceled)

12. The glass of claim 1, wherein the glass comprises: 48-50% SiO₂; 0.5-15% P₂O₅ (e.g. 10%); a combined molar percentage of CaO and SrO of 30-41%; a combined molar percentage of Na₂O and K₂O of 6-8% and 0.1-10% of a source of hypoxia mimicking ions.

13. The glass of claim 12, additionally comprising 4-6% ZnO and 4-6% MgO.

14. (canceled)

15. The glass of claim 1, wherein the glass comprises 47-62% SiO₂; 0.5-15% P₂O₅; 0-2% B₂O₃; a combined molar percentage of CaO and SrO of 12-17%; a combined molar percentage of Na₂O and K₂O of 12-27%; 0-6% ZnO; 0-3% MgO and 0.1-10% of a source of hypoxia mimicking ions.

16. (canceled)

17. The glass of claim 1, wherein the glass comprises: 47-50% SiO₂; 0.5-15% P₂O₅; 0-2% B₂O₃; a combined molar percentage of CaO and SrO of 18-21%; a combined molar percentage of Na₂O and K₂O of 24-27%; 0-6% ZnO and 0.1-5% of a source of hypoxia mimicking ions.

18. (canceled)

19. The glass of claim 1, wherein the glass comprises: 30-50% SiO₂, 0.5-15% P₂O₅; a combined molar percentage of CaO and SrO of 22-28%; 22-28% Na₂O and 0.1-5% of a source of hypoxia mimicking ions.

20. The glass of claim 1, wherein the glass is a bioactive glass.

21. The glass of claim 1, wherein the glass is not bioactive.

22. The glass of claim 2, wherein the combined molar percentage of SiO₂, P₂O₅ and B₂O₃ does not exceed 80%.

23. The glass of claim 2, wherein the combined molar percentage of SrO, CaO, MgO, Na₂O and K₂O is 40-60%.

24. The glass of claim 1, wherein the glass comprises 0.5-1.5% P₂O₅.

25. (canceled)

26. A 3D porous scaffold, fibre, coating implantable material, bone substitute, tissue regeneration construct, wound healing device, tissue engineering scaffold, suture, prosthetic implant, polymer matrix, fibrin gel, hydrogel, plaster, wound dressing, cream or a shampoo comprising a glass wherein the glass comprises:
30-62% SiO₂;
0.5-15% P₂O₅;
a combined content of CaO and SrO of 12-45%;
a combined content of Na₂O and K₂O of 6-30%; and
0.1-10% of a source of hypoxia mimicking ions,
wherein the hypoxia mimicking ions are selected from one or more of Co, Cu, Mn, Ni and Fe ions.

27-32. (canceled)

33. A composition comprising a glass of claim 1, wherein the composition is, a dental composite, a degradable polymer, a polymer scaffold composite, a natural polymer composite, a polymer suture, a bioactive porous scaffold, a wound dressing, a polypeptide gel, alginate beads, a bone substitute, a hydrogel, a fibrin construct, a powder, a cream, a shampoo, a bioactive glass filled acrylic, glass filled polyactide or other polymer, glass filled Bis GMA or dental composite, glass granules or a sintered glass.

34. (canceled)

35. A composition comprising a glass of claim 1 and cultured cells or cells directly isolated from a patient or donor.

36-37. (canceled)

38. A method of preventing and/or treating damage to a tissue comprising administering the glass of claim 1 to a patient in need thereof.

39. The method of claim 38, wherein the damage is chosen from soft tissue damage, bone fractures, periodontal disease, ulcers, burns, wounds, pressure sores, dental cavities, and cartilage damage.

40. (canceled)

41. A method of promoting bone or hard tissue repair or regeneration comprising administering the glass of claim 1 to a patient in need thereof.

42. A method of promoting hair growth and/or increasing hair thickness comprising administering the glass of claim 1 to a patient in need thereof.

43. A method of treating acne or infection comprising administering the glass of claim 1 to a patient in need thereof.
